(19) United States
(12) Patent Application Publication
(10) Pub. No.: US 2007/0243267 A1
(43) Pub. Date: Oct. 18, 2007

(54) Zeolitic compositions inhibiting bacillus anthracis toxins

(76) Inventors: Blanka Taborsky Barnett, Fuquay Varina, NC (US); Jiri Taborsky, Palmetto, FL (US)

Correspondence Address: Charles A. McClure, P. O. Box 1369, Lexington, VA, 24450, US

(21) Appl. No.: 11/401,748

(22) Filed: Apr. 12, 2006

(51) Int. Cl. A61K 33/06; C01B 33/36

(52) U.S. Cl. 424/684; 423/713

(57) ABSTRACT

Methods of preparing and using natural or synthetic zeolitic compositions therapeutically to alleviate, cure, or even preclude human host indisposition from exposure to bacteria within the Bacillus anthracis group: namely, B. anthracis, B. cereus, B. Mycoides, and B. thurigiensis. Exposure to bacilli of the first member of that group causes the often fatal disease anthrax.

### TECHNICAL FIELD

This invention relates to inhibition of biocatalysis, especially the inhibition of bacterial and viral enzyme activators, via interrelations of enzymes, ions, and zeolites or equivalent ion-exchanging compositions, with special application to bacteria of the Bacillus anthracis group.

### BACKGROUND OF THE INVENTION

As stated by Bohinski, in Modern Concepts in Biochemistry, “the totality of cellular activity is intimately dependent on the type and concentration of ionic materials within the cell, both of which are subject to change by alterations in the extracellular environment.” [1] As stated by Dressler and Potter in Discovering Enzymes, “Not to put too fine a point on it, enzymes control all of the chemical transformations in the living world.” [2]

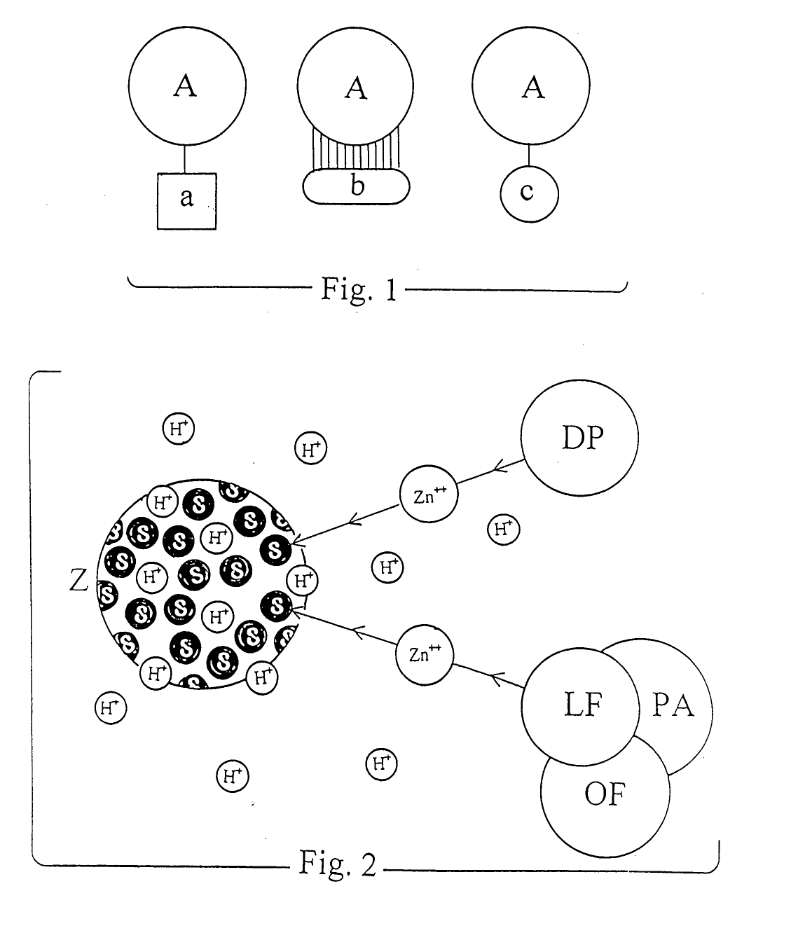

Enzyme-controlled reactions are essential to all phenomena of life. Nearly every cellular activity is catalyzed by enzymes, many of the enzymes dependent upon associated cofactors. Though differences between those cofactors may not be sharp-edged, Holum's proposition [3] lists three generally accepted categories of cofactors (also see a schematic thereof in FIG. 1), thus: a., a coenzyme: a non-protein organic substance (e.g., a vitamin) dialyzable, thermostable, and loosely attached to an apoenzyme; a true substrate for enzyme-catalyzed reaction, recycled in a later step of a metabolic pathway by another enzyme; b, a prosthetic group, a dialyzable and thermostable organic substance, firmly attached (vertical lines) to the protein of the apoenzyme portion; and c, a metal cation activator, metal cations being critical to enzyme function, structure, and stability.

In general, the more complex an organism, the more complex and numerous its enzymes, and the more likely it can survive some enzymatic irregularity, such as inadequate concentration, or absence of a given enzyme. Whereas metabolism in vertebrates depends upon a vast number of enzymes, whose activity may require presence of other enzymes, coenzymes, or similar cofactors, more primitive life forms (e.g., viruses, bacteria, protozoa, fungi) survive with fewer enzymes, often controlled only by an ion activator, and may have their metabolism or replication terminated by dysfunction of a single enzyme. The present invention views enzymes as targets, with objectives of neutralizing pathogens, controlling development of neoplasms, etc.—with minimal collateral damage to their respective hosts.

Current methods of inhibiting microbial enzymes rely mostly upon activity of chemical agents; limited in degree upon such other means as heat treatment, radiation, immunization, hormone application, or genetic engineering. However, all of these approaches often have severe limitations and/or serious side effects. Researchers focus upon chemical enzyme inhibitors, usually antibiotics or other organic chemicals, administered to the host organism, and often causing eventual deleterious side effects. Some of them interfere with cell division, and often are toxic to both host and invader cells, while many of them may contribute to development of resistant mutation of targeted and/or non-targeted pathogens.

The present invention directs attention to adsorption of ions necessary for biocatalysis, via ion-exchangers or sorbents, such as hydrous aluminosilicate compositions, here exemplified specifically by zeolites, which may be natural or synthetic. Both natural and synthetic zeolites are well known as sorbents, carriers and ion-exchangers of ionic substances often intended to catalyze or to inhibit certain chemical activity.

Sometimes zeolites are used, either solitary or distributed within an organic polymer, to convey a toxin, a chelate, or a heavy metal cation as a bactericide or fungicide, as in cosmetics and medicines. See, for example, Yoshimoto et al. U.S. Pat. No. 4,870,107 (1989); Hagiwara et al. U.S. Pat. No. 4,775,585 (1988), U.S. Pat. No. 4,911,898 (1990), U.S. Pat. No. 4,959,268 (1990), Satoshi et al. Japanese Patent Application 03218916 A (1991); Satoshi et al. Japanese Patent Application 03255010A (1991); and Wagner U.S. Pat. No. 4,824,661 (1989). In Chu et al. U.S. Pat. No. 5,140,949 (1992), for example, a mixture of zeolite and clay is proposed as a feed supplement, and as a topical treatment, based on its ability to adsorb ammonium cations. Similarly, in Polak et al. U.S. Pat. No. 5,409,903 (1995), zeolite alone, or zeolite in a mixture of other chemicals, is proposed for the treatment of Helicobacter pylori and dermatitis. U.S. Food Additive Regulation 582-2727 approves zeolite use in feeds as an anti-caking agent, and USDA approves them as sorbents in food processing applications; being in EPA compliance (40 CFR, Part 180.1001 and elsewhere): Engler U.S. Pat. No. 5,900,258 features silicates, de-aluminated but neither deionized nor homo-ionized, to inhibit microorganism growth on and within textile and other interstitial or porous materials, also on relatively impervious extensive structural or working surfaces, and in nutrient material fed to chickens in order to evaluate its possibility for reducing incidence of microorganisms in or arising from such feed. All of the foregoing efforts are of minor interest. Other specific uses of zeolites as carriers of substances harmful to biological, sometimes enzyme-dependent, activity also could be cited, but also are distinct from the present invention.

### SUMMARY OF THE INVENTION

A primary object of the present invention is inhibition of bacteria of the Bacillus anthracis group and their toxins.

Another object of this invention is to inhibit any biotype, serotype or other induced or spontaneous mutation of bacteria of the B. anthracis group, including antibiotic-resistant strains.

A further object of the invention is to deactivate the toxins and any mutation of toxins of the B. anthracis group (an objective that cannot be achieved by antibiotics.).

Yet another object of this invention is to provide effective means of prophylaxis, to limit likelihood of infection from contaminated air, liquids, foodstuffs, bodily surface contact, etc.

A still further object of the present invention is to accomplish the foregoing objects in an economically sound way and in a manner safe to the human organism.

In general, the objects of this invention are achieved by inhibiting activity of microbial enzymes and microbial metal-dependent toxins, by supplying to the site of that activity a sorbent or ion-exchanger, specifically a properly constituted zeolite, effective to adsorb ions and related substances provocative of such undesirable biochemical activity.

By a synergic action, zeolites appropriately selected, such as to density and size of pores, are adapted to serve as molecular sieves to bind an entire holotoxin. Adsorption of toxins, as an ability of zeolites, is a practical expedient often resorted to in the substance-separation industries.

More particularly, the objects are attained by inhibiting the action of zinc-dependent bacterial protease, an essential to bacterial metabolism within the B. anhracis group, and by inhibiting the zinc-dependent lethal factor (LF) of that group's tripartite toxin. Deionized zeolite may well simultaneously inhibit adenylate cyclase (toxin's OF).

Alteration in relative affinities of natural zeolites for given monovalent and divalent cations, by dry heating pretreatment, and the benefits of doing so are disclosed in Taborsky U.S. Pat. Nos. 5,082,813; 5,162,276; and 5,304,365. Zeolites or equivalent compositions may be ion-pretreated, e.g., by deionization, or by homo-ionization, or may be synthesized in specific (e.g., hydrogen) cation form, and be applied as a broad-range sorbent, or may be selectively reionized for specific applications. Equivalent compositions may be combined for complementary and/or synergic purposes and/or for their affinities for ions or classes thereof.

Other objects of the present invention, together with means and methods for attaining the various objects, will become readily apparent from the following description, presented by way of example rather than limitation.

FIG. 1 comprises three schematized representations of a complete enzyme, comprising an apoenzyme (A) in separate conjunction with each of several different cofactors: (a, b, c).

FIG. 2 is a schematized representation of a zeolite (Z), in conjunction with each of a zinc-activated protease (DP) and a tripartite toxin (LIF+OF+PA) as in a digestive enzyme.

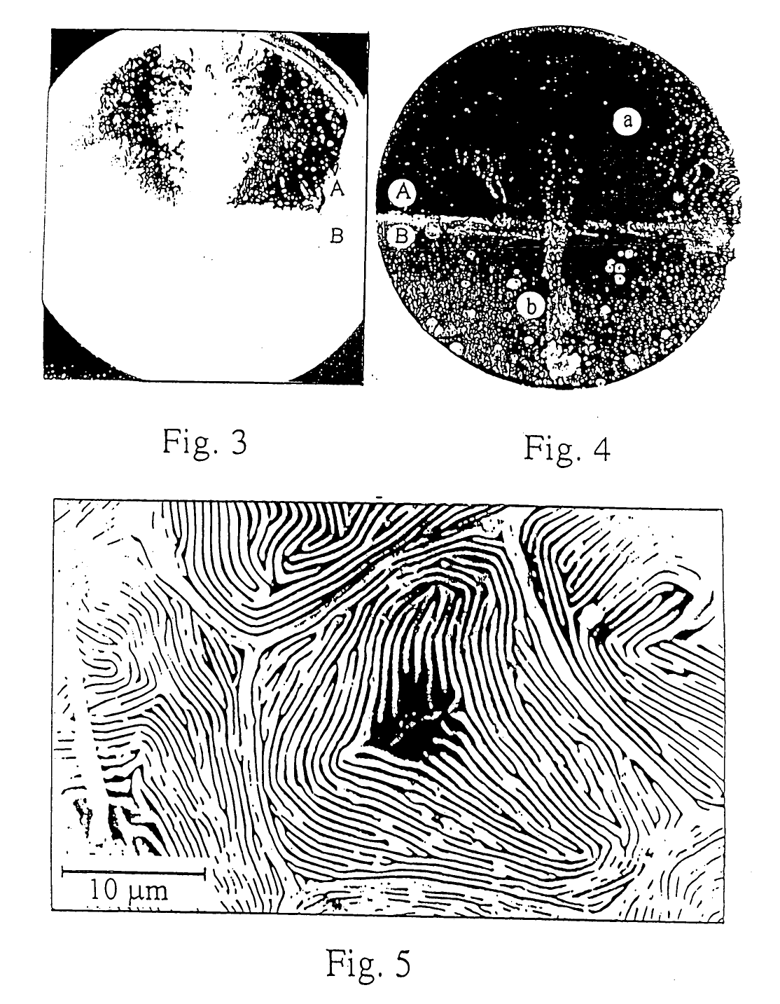

FIG. 3 shows a Petri dish with untreated upper half (A) darkened by micro-colonies growing on agar, and with other (lower) half (B) clear, growth being precluded by added zeolite.

FIG. 4 shows a Petri dish also with light untreated half (A) and darkened treated half (B).

FIG. 5 is a scanning-electron photomicrograph of squamous epithelial gas-exchange cells.

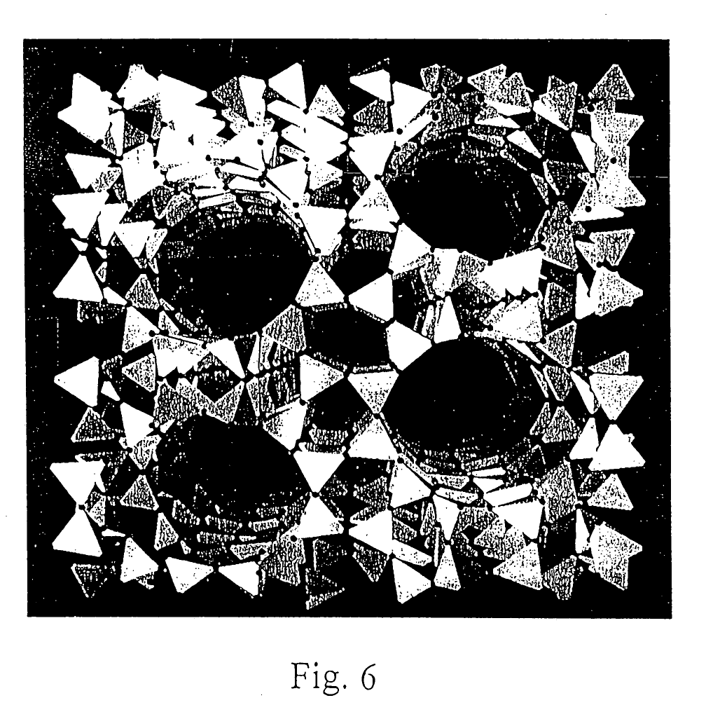

FIG. 6 is a computer-generated image of a zeolite showing its porous crystalline structure.

### DESCRIPTION OF THE INVENTION

The invention is characterized in practical terms, so as to enable its successful practice, regardless of any academic or theoretical conceptualization expressed in this exposition thereof, as concurrence in the latter is not a prerequisite for successful practice of the actual invention.

Whereas “ionization” generally means a process of producing ions, in this description “ionization” and its inflected forms (e.g., reionization, homo-ionization) have the meaning of charging or loading a zeolitic adsorbent or similar ion-exchanger with ions—as a logical opposite of the unambiguous term “deionization” (being a conventional term for removing ions). Preliminary Testing of inhibition of enzyme activity by zeolitic adsorption was conducted using the accepted ninhydrin (1,2,3-triketohydrindene hydrate) test for presence of amino acids from enzymatic breakdown of caseins follows.

1. At room temperature, 100 mg of bacterial protease was stirred into 100 ml of distilled water containing 10 g of deionized (method 2 Bd) clinoptilolite particles (<74 μm). After about 10 minutes of mild agitation, 10 ml of this solution was stirred thoroughly into 50 ml of 5% DIFCO isoelectric casein solution, which tested negatively as to free amino acids an hour thereafter. This test indicated that the bacterial protease was deactivated.

2. To avoid any mistake caused by an eventual interaction between casein and zeolite, the test was modified as follows: at room temperature, 100 mg of bacterial protease was stirred into 100 ml distilled water containing 10 g of deionized (method 2 Bd) clinoptilolite particles (300>600 μm). After about 10 minutes of mild agitation, the suspension was filtered through a 200 μm nylon sieve, then 10 ml of the filtrate was stirred thoroughly into 50 ml of 5% DIFCO isoelectric casein solution, which then tested negatively for free amino acids an hour thereafter. This result indicated that the bacterial protease was deactivated.

The zeolite adsorbed cations from the enzyme and inhibited it from breaking the casein down and providing amino acids for detection. Similar tests conducted with different species of zeolite (phillipsite, chabazite), with deionized samples from different sources, and with synthetic zeolites (Y, Beta, and ZMS-5 powders), and all test results indicated inhibition of the enzymes.

Further analogous tests showed that all previously used deionized natural and synthetic zeolites inhibited all tested bacteria, but tests with virgin natural zeolites and H⁺ homo-ionized zeolites were not entirely conclusive, suggesting a need for pretesting of each batch thereof, or for limiting actual operations to use of zeolites pretreated as specified here, operative both in vitro and in vivo, such as for agriculture, dentistry, medicine, and biological instances generally. Selection, modification, and applicability of zeolites.

The aluminosilicates known as zeolites are customarily used as ion-exchange media, in effecting separation and recovery of dissolved materials, liquids and gases, as carriers of ions, and specifically as molecular sieves (e.g. for cracking petroleum fractions). Aluminosilicate minerals occur in many geographical locations and include prominently zeolites: clinoptilolite, heulandite, chabazite, phillipsite, analcite, brewsterite, faujasite, ferrierite, flakite, gmelinite, leucite, stilbite, and yugawaralite; also the layer (or pseudo-layer) silicates, vermiculites and smectites—often called layered clays.

The foregoing minerals are hydrated mixed aluminosilicates, with compositions determined largely by the constituents available when they were formed, resulting in diverse crystalline structures. Synthetic aluminosilicates have been produced with more controlled compositions, and often are designated by a letter (e.g., “F”, “X”) appended to “zeolite.” Whether produced under laboratory conditions or in mineral deposits, aluminosilicates range widely in composition and physical properties. Their identification, as well as. their properties, can vary, depending upon specific interesting characteristics—here, their physical properties, modification and manipulation of accessible surfaces and sites for adsorption of microbial enzyme activators.

Zeolites have a distinctive molecular arrangement causing a negative charge of their molecules. It results in strong adsorptive power, unmatched by any other adsorbent and strong enough to penetrate the protective coats of vegetative forms of microbes, and moist-swollen exosporia, and protective coats of endospores. A similar transfer of cations through gels has been well demonstrated (see section 7B below: Application of zeolitic enzyme inhibitor). Zeolites form extremely porous crystalline conglomerates having tiny uniform pores, measuring in some species only a few A, and endowing them with tremendous interior surface area having numerous ion-exchangeable sites. Their negative charge enables these sites attract, bind, and eventually exchange, cations.

Consequently, zeolites possess a unique ability to adsorb metallic activators of enzymes controlling biochemical processes in viruses, bacteria and some other low-organized organisms. Both natural and synthetic zeolites were tested in practicing and evaluating this invention.

Zeolites, when properly selected and modified, or synthesized, are, for all practical purposes, chemically inert and do not cause any chemical side effect to the host organism. They are not recognized by the pathogen or by the host as xenobiotics. Therefore, they are unlikely to trigger any immunologic reaction in the host or to activate any defense mechanism of the pathogen. Hence, the pathogens are unlikely to develop any resistance to the loss of enzyme activator, as in the practice of the present invention.

Zeolites (and similar sorbents or ion-exchangers) work in three principal ways, without any appreciable toxic or biochemical impact on the host organism: (a) inhibiting activity of microbial metalloenzyrnes; (b) deactivating toxins; and (c) adsorbing cations from immediate microenvironments, thus preventing microbes from utilizing them for replenishment or for production of new metalloenzymes.

Virgin natural zeolite species exhibit substantial differences in chemical composition, crystalline structure, density, and levels of impurities. Zeolites of high density, zeolites with a considerable crystalline silica contamination, fibrous zeolites, and zeolites contaminated with specific cations, and the like, are unsuitable for medical purposes. Accordingly, deionized, homo-ionized, selectively ion-recharged, or otherwise modified natural and synthetic zeolites are preferable to virgin zeolites in the practice of this invention.

Specific zeolites, especially modified and synthetic ones, also exhibit pH differences, so the pH of zeolites for sensitive applications should be appropriately pre-adjusted. Yet, to maintain the pH value in a narrow range at the site of application is often impractical, owing to sorption of atmospheric carbon dioxide, metabolic activity of organisms, and other factors.

1. Adsorbants used in experiments:

A. Natural zeolite: clinoptilolite, hydrated sodium potassium calcium aluminum silicate (Na, K, Ca)₂O.Al₂O₃.10SiO₂.8H₂O, Winston, N. Mex. deposit, 4×6 size granules (approx. 5 mm).

Analysis (weight % for major oxides): Bowie and Barker, NM Bureau of Mines, 1986)

| | |
|---|---|
| Silicate | 64.7 |
| TiO₂ | 0.2 |
| CaO | 3.3 |
| K₂O | 3.3 |
| MnO | 0.1 |
| Fe₂O₃ | 1.8 |
| Al₂O₃ | 12.6 |
| Na₂O | 0.9 |
| MgO | 1.0 |

Chemical Composition for given elements, by x-ray fluorescence (ppm, or wt. % noted):

| | |
|---|---|
| K | 2.0% |
| Fe | 0.9% |
| Ce | 90 |
| Cu | 30 |
| Rb | 70 |
| Pb | 40 |
| Zr | 190 |
| Nb | 20 |
| Nd | 15 |
| Ba | 1030 |
| Ca | 2.7% |
| Sr | 1720 |

(Desborough, USGS OF Rpt 96-065 & 265, 1996.)

Cation Exchange Capacity:

1.00-2.20 meq/g (may vary, as CEC values are relative to procedure and specific cations). Major Exchangeable Cations, Rb⁺, Li⁺, K⁺, Cs⁺, NH4⁺, Na⁺, Ag⁺, Ca⁺⁺, Cd⁺⁺, Pb⁺⁺, Zn⁺, Ba⁺⁺, Sr⁺⁺, Cu⁺⁺, Hg⁺, Mg⁺⁺, Fe⁺⁺, Fe⁺⁺⁺, Co⁺⁺, Al⁺⁺⁺, Cr⁺⁺⁺, Mn⁺⁺⁺. (Selectivity of such cations is a function of-hydrated molecular size % relative concentrations).

Purity:

Analysis by x-ray diffraction at the N M. Institute of Mining and Technology and other tests suggest an 80% clinoptilolite content with the remaining material primarily inert volcanic ash and sediments. Clay and other mineral varieties are detectable only in minute quantities.

Physical Properties:

| | |
|---|---|
| pH (natural) | 8.0 (approx.) |
| Acid Stability | 0-7 (pH) |
| Alkali Stability | 7-13 (pH) |
| Bulk Density (In Place, dried) | 87 lbs/ft³ (1,390 kg/m³) |
| Bulk Density (Aggregate, dried) | 44-48 lbs/ft³ |
| −40 Mesh | 783-1054 kg/m³ |
| Cation Exchange Capacity (CEC) | 1.0-2.2 meq/g |
| Color | White (85 optical reflectance) |
| Crushing Strength | 2500 lbs/in³ (176 kg/m³) |
| Hardness | 3.5-4.0 Mohs |
| LA Wear (Abrasion Index) | 24 |
| Mole Ratio | 5.1 (SiO₂/Al₂O₃) |
| Other | non-soluble, non-slaking, free |
| | flowing |
| Pore Size (diameter) | 4.0 Å |
| Pore Volume | 52% (max.) |
| Resistivity | 9,000 (approx.) ohms/cm |
| Specific Gravity | 2.2-2.4 |
| Surface Area | 1357 yd2/oz (40 m²/g) |
| Swelling Index | nil |
| Thermal Stability | 1202° F. (650° C.) |

B. Synthetic zeolite: Zeolyst® Y Type zeolite powder (FAU) CBV 400 in cation form.

| | |
|---|---|
| Mole Ratio | 5.1 (Si₂O/Al₂O₃ |
| Surface area | 730 m²/g |
| Unit Cell Size | 24.50 Å |

2. Modification of Adsorb Ants:

A. H⁺ Homo-Ionization:

A solution of substantially any salt (e.g., NaCl) or any hydroxide (e.g., NabH) or some combination of them may be used for selective homo-ionization or polyionization of zeolites, depending upon a specific application. H⁺ homo-ionization alone might not be a sufficient pretreatment for medical purposes (but see exception described in part 2Aa). Thus, zeolites for the purposes of the present invention should be so pre-ionized by monovalent cations to which the zeolite has high affinity (e.g., Na⁺ or K⁺) as described in part 2Ad below.

a. H⁺ Homo-Ionization via Ammonia Decomposition:

The zeolite is first impregnated by -ammonium cations (preferably using ammonium hydroxide) to displace the achievable maximum of other cations via ion-exchange, then washed, and finally heated to 500° C. At this temperature, ammonium decomposes to gaseous ammonia, which escapes, and hydrogen cations, which occupy available adsorption sites of the zeolite. The thermal stability of treated zeolite species and types must be considered unless a distortion of crystalline structure is negligible for the given application, or if a certain distortion of the crystalline structure is desirable as an additional functional modification.

b. H⁺ Homo-Ionization Via Electrolysis of Water:

A stream of hydrogen cations generated by electrolysis of water is directed through a bed, preferably a fluid bed, of sand or granule-sized zeolite. Via ion-exchange, an achievable maximum of other cations on ion-exchangeable sites is replaced by hydrogen cations. The excess of hydrogen cations is reduced on the cathode to hydrogen gas. The cathode should be placed in a trapping devise that collects reduction products and positively charged impurities. While this is an elegant and very pure method, an eventually insufficient concentration of electrolyte may render the H⁺ homo-ionization imperfect. The performance of the process may be improved by a modification of the electrolytic apparatus, provided by rotating chambers, by rotating phases of polarity, and/or by enhancing the electrolyte with ionized effluent water from one (or more) auxiliary electrolyzer(s). Such modified electrolytic apparatus also may be applied advantageously for ionization, homo-ionization, or reionization processes described later.

c. H⁺ Homo-Ionization Via Acid Treatment:

Most acids are suitable, but inorganic acids are preferable, especially nitric acid because of easy disposal of NO₃ anions. Zeolite of sand or granule size is treated with diluted acid in order to displace the achievable maximum of other cations by hydrogen cations via ion-exchange, then rinsed with deionized or distilled water to remove formed salts and anions to an achievable minimum.

d. Combined Homo-Ionization:

Generally, zeolites have a weak affinity-for hydrogen ions. Consequently, hydrogen ions cannot completely displace the more preferred cations (erg., K⁺, Na⁺, Zn⁺⁺, Mg⁺⁺) by simple ion-exchange. Hence, the zeolite should first be homo-ionized by monovalent cations with strong affinity (preferably Na⁺) and then be H⁺homo-ionized (as already disclosed) to a maximally achievable degree. An eventual Na⁺ residue does not interfere with the inhibition of the BD anthracis group of bacteria.

Unless a synergic low pH effect is sought, a direct application of H⁺ homo-ionized zeolites is unsuitable for certain microbe treatments in vivo because the extremely low pH has an adverse effect on human, especially epithelial, tissues, possibly excepting gastrointestinal applications when zeolites are H⁺ homo-ionized in the stomach anyway and then gradually deionized to H⁺ equilibrium in the duodenum. In in vitro applications, the H⁺ homo-ionized zeolites contaminate the culture media with hydrogen cations and render experimental results less reliable. Most importantly, hydrogen cations (occupying ion-exchangeable sites) interfere with instant adsorption of other cations including the enzyme activators by causing a delay in the ion-exchange and in the establishment of equilibria.

B. Deionization:

a. Partial:

The zeolite is washed with deionized or distilled water until most ion-exchangeable sites are, by equilibration, free of cations. The achievable maximum of partial deionization is given by cation/zeolite equilibrium. For achieving high degree of deionization, this method is too time-consuming and is economically unfeasible.

b. By Water Electrolysis:

A bed, preferably a fluid bed, of zeolite is first electrolytically H⁺ homo-ionized (method 2Ab), rinsed, and then the polarity of electrodes is reversed, whereby the zeolite bed is exposed to a stream of hydroxyl anions (OH⁻) until the desired pH is stabilized.

c. By a Combination Method:

A bed, preferably a fluid bed, of zeolite, already H⁺ homo-ionized (method 2Aa, 2Ac) is exposed to a stream of hydroxyl anions (OH⁻) until the desired pH is stabilized.

d. By (OH⁻) Effluent from an Anion Exchanger:

A bed, preferably a fluid bed, of H⁺homo-ionized zeolite is exposed to an OH⁻ water effluent generated by an anion-exchanger until the achievable maximum of hydrogen cations has been removed from the zeolite (or until a desired pH is established), via formation of water during the process of equilibration.

C. Selective Ionization:

For specific purposes (e.g., to prevent or to mitigate adsorption of selected cations, such as calcium or iron cations, or to deliver to the site cations for specific purposes (e.g., copper or cobalt ions) zeolites may be ionized with any selected metal cation or cations via appropriate salts or hydroxides. A selective ionization may be imlplemented during or immediately after homo-ionization, or instead of homo-ionization. The following methods are preferred in the practice of this invention.

a. Selective Reionization by Cations Toxic to Bacteria:

Using otherwise untreated (virgin) zeolites as carrier of toxic cations to function at a destination site is known. However, for medical purposes the prior art is generally unsuitable because the concentration of such toxic cations in virgin zeolites is difficult to establish and maintain because of uncontrollable factors, such as impurities, present unavoidable cations, pH fluctuation and consequent fluctuation of toxicity level, and equilibria in the microenvironment and within the zeolite. In many applications accuracy in the cation concentration is critical. For example, excessive concentrations of copper cations will mitigate or completely inhibit production of mucous surfactant, which protects the host's gas-exchanging cells [4][5], and thereby may cause irreversible damage to a host's respiratory system and eventually result in death of the host, The methods of pretreatment of zeolites as disclosed in this application, especially in applications of deionized zeolites, allow appropriate accuracy of the dosage of toxic cations, thereby protecting the host's epithelial tissues, and equalizing any eventual site competition. For present purposes, copper cations were preferable because of their valence identity to the targeted zinc cations of B. anthracis group enzymes, thereby equalizing any eventual site competition, and because the present inventors have ample experience with the effects of copper ions upon bacteria and vertebrates. Deionized clinoptilolite was batch-enhanced with 0.05′ weight percent (chosen from an eventual range of 0.001-3.0%) by direct adsorption from a copper sulfate solution.

b. Selective Ionization of Zeolitic Inhibitor by Cobalt Cations:

Selective ionization by cobalt cations is of special interest. One of the severe symptoms of inhalational anthrax is shortness of breath. It is caused (besides the bacterial damage to the alveolar epithelium) by the consumption of zinc ions by anthrax bacilli. Zinc cation is also the primary activator of carbon anhydrase—the enzyme catalyzing the reversible hydration of CO₂ to H₂CO₃, a necessary reaction for facilitation of transport of CO₂, and transfer and accumulation of H⁺ and HCO₃. Hence, the deficiency of zinc cations contributes substantially to the inhibition of the respiratory gas-exchange process. However, some carbon anhydrases are able to-function with an alternative metal activator, as with the cobalt cation in this instance [19]). Zeolitic inhibitor adsorbing zinc activators from bacteria, from their immediate macroenvironment, and from their toxins, can serve simultaneously as a carrier of cobalt cations to boost the carbon anhydrase catalytic activity, and thereby greatly mitigate the “short breath” symptoms. The cobalt cations may be administered via a zeolitic carrier, or as a part of a compound (e.g., salt or chelate) in any suitable therapeutic form (e,g,. aerosol, hydrosol, intravenous infusion, or extracorporeal filtration of bodily fluids through a cobalt-impregnated zeolitic bed).

3. Bacteria of the Bacillus anthracis group:

Within the genus taxon, the current taxonomic and nomenclatoric rules do not recognize any “group” taxon—which is only ancillary, indicating a close systematic and phylogenetic relation of certain species, here those of B. anthracis, namely: B. cereus, B. mycoides and B. thuringiensis. This group is frequently informally designated as the Bacillus cereus group. See, Genus Bacillus Cohn 1872. Hierarchy: Monera-Bacteria-Inside series of Bacteria-Bacillales. Nomenclatural/taxonomic status: Approved Lists Type species: B. subtilis Reference(s): Int. J. Syst. Bacteriol. 30:256 (AL), (Bergey's manual of determinative Bacteriology, 8th ed., 1974; Editors: Buchanan, R. E., Gibbons, N. E; Publisher: The Williams & Wilkins Co., Baltimore).

The B. anthracis group is a group of closely related species within the genus Bacillus. Though classified as valid different species, these organisms seem to differ only in the plasmids. All four species are large straight rod-shaped Gram-positive, non-flagellated, endospore-producing bacteria, whose spores do not swell the sporangium. They are often aerobic cells of 1-10 μm in length, and 1-1.5 μm in breadth, with a “jointed bamboo-rod” cellular appearance.

All species of the B. anthracis group are pathogenic to humans, causing known or potential cutaneous/subcutaneous, intestinal, inhalational and other infectious conditions. The endospores are approximately 1 μm, species-indistinguishable within the group. Endospores are extremely resistant and may survive, for entire geological periods, at temperatures ranging from absolute zero to −40° C., and for decades between −30° C. and at least 50° C. They can withstand several minutes of usual autoclave sterilization and at least one minute of usual microwave sterilization. They germinate readily, and their vegetative cells grow on all ordinary laboratory media, at like temperatures and times, except that B. anhthracis prefers a range closely about 37° C. Bacteria of the B. anthracis group share a multitude of other characteristics, including both biochemical and biophysical properties. Differentiation of the respective organisms is done in the vegetative form by determination of motility (B. cereus rods are usually motile), and by the presence of toxin crystals (B. thuringiensis), and also by hemolytic activity (B. cereus and B. thuringiensis are beta-hemolytic, B. anthracis is usually non-hemolytic), by growth requirement for thiamin, by lysis via gamma phage, by growth on chloral-hydrate agar, and further by the morphology of micro-colonies (e.g., a rhizoid growth is characteristic for B. mycoides [see FIG. 3], and a perloid growth pattern for B. anthracis).

A. Bacillus anthracis (Cohn 1900), various synonyms: Bacillus cereus var. anthracis (Cohn 1872) Smith et al. 1946; Bacteridium anthracis (Cohn. 1872) Hauduroy et al. 1953. Nomenclatural/taxonomic status: Approved Lists Reference(s): Int. J, Syst. Bacteriol. 30:256 (AL), Ref.: Bergey's manual of determinative Bacteriology, 8th ed., 1974; Editors: Buchanan, R. E., Gibbons, N. E; Publisher: The Williams & Wilkins Co., Baltimore); Risk group: 3 (German classification) Type strain: ATCC 14578. Bacterial proteolytic enzyme: Zn⁺⁺ activated protease; LF of the tripartite toxin: specific Zn⁺⁺ activated protease; OF: adenylate cyclase.

Bacillus anthracis is usually an aerobic, nonmotile species. The vegetative cells are large rods (1-8 μm long, 1-1.5 μm wide). B. anthracis is the causative agent of the anthrax disease. The symptoms of all three forms, cutaneous, intestinal, and inhalational, are well known [15]. Anthrax has been intended, as one of the most dangerous biological warfare agents for more than eighty years. Within that time, countless deadly serovars have been developed, many of them as antibiotic-resistant and drug-resistant strains. Neither trials nor any cultivation of B. anthracis were conducted for purposes of this invention, but experimentation on other of the members of the group has been undertaken successfully and tentatively is believed to be applicable to every B. anthracis group member, based upon their close phylogenetic relationship.

B. Bacillus cereus (Frankland & Frankland 1887) ambiguous synonym(s): Bacillus cereus var. anthracis, Bacillus thuringiensis, Bacillus endorhythmos, Bacillus medusa. Nomenclatural/taxonomic status: Approved Lists Reference(s): Int. J. Syst. Bacteriol. 30:256 (AL), (Ref.: Bergey's manual of determinative Bacteriology, 8th ed., 1974; Editors: Buchanan, R. E., Gibbons, N. E; Publisher: The Williams & Wilkins Co., Baltimore); Risk group: 2 (German classification) Type strain: ATCC 14579, CCM 2010, NCIB 9373, NCTC 2599. Bacterial proteolytic enzyme: Zn⁺⁺ activated protease; LF of the tripartite toxin: specific Zn⁺⁺ activated protease; OF: adenylate cyclase. Intestinal infection, causing food poisoning, has been believed for a long time to be the only medical concern. The symptoms of the diarrhea type of food poisoning mimic those of Clostridium perfringens, beginning with watery diarrhea, sometimes accompanied by nausea and vomiting. Abdominal pain and cramps occur 6-15 hours after infection. Usually such symptoms persist for 24 hours. Symptoms of the emetic type are similar to those of Staphylococcus aureus: nausea and vomiting within 30 minutes to 6 hours after consumption of contaminated food. Abdominal cramps and diarrhea may occur too. Symptoms generally last less than 24 hours.

Recently, however, cutaneous B. cereus infections causing acute necrosis very similar to the cutaneous form of anthrax have been reported. Even more dangerously, several cases of B. cereus infections of other tissues occurred: including rapidly fatal meningoencephalitis [14], septicemia, mastitis, and several cases of potentially blinding endophthalmitis [6][7].

Since B. cereus is a typical airborne-spore proliferater, and sporulates and germinates easily, it is a potential agent for inhalation infections. No verified case of an inhalational form of infection has been reported yet. It may be hypothesized that B. cereus OF enzyme did not mutate yet—as B. anthracis did to be effective enough to impair the host's defense system.

Inocula: Bacillus cereus strain CBSC 15-4870/2001, freeze-dried CBSC 15-4870A/2001.

C. Bacillus mycoildes (Flügge 1886), ambiguous synonym: Bacillus mycoides corallinus. Hefferan 1904. Nomenclatural/taxonomic status: Approved Lists Reference(s): Int. J. Syst. Bacteriol. 30:257 (AL), (Ref.: Bergey's manual of determinative Bacteriology, eighth ed., 1974. Editors: Buchanan, R. E., Gibbons, N. E; Publisher: The Williams & Wilkins Co., Baltimore; Die Mikroorganismen, 3rd ed. vol. 2, 1896; Editor: Flugge, C.; Publisher Vogel, Leipzig); Risk group: 1 (German classification); Type strain: ATCC 6462. Bacillus mycoides is in almost all of its characteristics like B. cereus—but for its morphological rhizoid pattern of micro-colonies. Bacterial proteolytic enzyme: Zn⁺⁺ activated protease; LF of tripartite toxin: specific Zn⁺⁺ activated protease; OF: adenylate cyclase. Inoculum: B. mycoides strain CBSC 15-4870/2001.

D. Bacillus thuringiensis (Berliner 1915) ambiguous synonym: Bacillus cereus var. thuringiensis (Berliner 1915) ambiguous synonym: Bacillus cereus var, thuringiensis (Smith et al. 1952). Nomenclatural/taxonomic status: Approved Lists Reference(s): Int. J. Syst. Bacteriol. 30:258 (AL) (Ref.: Bergey's manual. of determinative Bacteriology, 8th ed., 1974; Editors: Buchanan, R. E., Gibbons, N. E; Publisher: The Williams & Wilkins Co., Baltimore); Risk group: 1 (German classification); Type strain: ATCC 10792, Sp2000 Taxon Code: BIO-6867.

Bacterial proteolytic enzyme: Zn⁺⁺ activated protease; LF of the toxin: specific Zn⁺⁺ activated protease; OF: adenylate cyclase.

B. thuringiensis is a bacterium, marketed worldwide as a specifically targeting bioinsecticide for control of plant pests (mainly caterpillars of the Lepidoptera), for control of mosquito larvae, simuliid blackflies, etc. Genetic material from B. thuringiensis toxin is used in the development of genetically engineered corn, cotton, and other crop plants. Most BT insecticides are derived from genetically improved mutations of B. thuringiensis biovar isrqelensis or B. thuringiensis kurstaki. The active ingredients marketed BT products are the bacterial dormant spores (>10¹² per liter) and proteinaceous aggregates, including crystal-like parasporal inclusion bodies (PIB). The research done for manufacturers of BT products presents the bacterium as safe to human health. Yet, much as may be indicated, for example in DiPel® DF MSDS [16], the trials appear purpose-designed. In general, the health implications of exposures to B. thuringiensis, especially inhalational effects, have not been yet satisfactorily investigated.

Because of the close phylogenetic relation between B. anthracis group species, it should be taken into consideration that a dose of Bt spores, sufficiently potent to cause an inhalational Bt infection, may cause an infection with symptoms mimicking the symptoms of an anthrax infection (in at least one test, the mortality in guinea pigs was 10% [10]).

The BT products generate nonspecific cytotoxicities involving loss in bioreduction, cell rounding, blebbing and detachment, degradation of immuno-detectable proteins, and cytolysis. Some research data indicate that spore-containing BT products have an inherent capacity to lyse human cells in free and interactive forms and may also act as immune sensitizers [17].

Inocula used: Bacillus thuringiensis strain CBSC 15-4870/2001 (vegetative cells), CBSC 15-4870A/2001 (lyophilized vegetative cells), Javelin® (endospores, strain not identified), Thuricide® (endospores, strain not identified), and Skeetal® Abate (endospores, strain not identified).

Classified in the Bacillus anthracis group may be a newly described species Bacillus pseudomycoides but there has not been yet a sufficient research done to validate it.

Bacillus pseudomycoides Nakamura 199 8 Reference(s): BIO-6840, Nakamura (L.K.): Bacillus pseudomycoides sp. nov., Int. J. Syst. Bacteriol., 1998, 48, 1031-1035. No trial nor any cultivation of B. pseudomycoides has been conducted for the purposes of this invention.

Many characteristics of B. anthracis, B. cereus, B. mycoides and B. thuringiensis are alike. For this invention the most important trait is that the bacterial metabolic protease and the lethal factor (LF) of the toxin are zinc-dependent; that is, the enzymes are activated by the zinc cation [8]][9][19][11][12][13]). Thus, it can be assumed with reasonable certainty that the mechanism of zeolitic inhibition of their metabolic proteases and the deactivation of their toxin enzymes is similar in-al four species, especially in B. cereus and B. anthracis, being so clearly alike in so many regards.

4. Microbiological Media

A multitude of suitable media for culturing Gram+ bacteria has been tried, including standard beef bouillon, nutrient gelatins and broths, count agar, modified nutrient agar (without peptone), protein-enriched nutrient agar, TSA w/5% and 10% sheep blood, etc. All media tested supported growth of vegetative cells and germination of endospores (where applied) along with the expected unimportant differences in morphological patterns of micro-colonies.

After preliminary testing for suitable uniform media, a T-011 modified nutrient agar was chosen, consisting of standard beef extract, 5 g; agar, 15 g; with rehydration, 23 g/1000 ml. At its pH of 6.8, this agar is well within the optimal range for the Bacillus anthracis group.

5. Inoculation of Bacteria

Many inoculation methods were tried in preliminary assays, including direct swab smear, diluted smears, loop inoculations in varied cell concentrations, smears and loops of inocula diluted in redistilled water, as well as smear and loop inocula diluted in physiol solution. Dry inoculates of spores were tried also (where applicable). In all trials, the temperature was maintained at approx. 25° C. All of these experimental methods proved satisfactory. After these preliminary trials, two specific methods were selected for formal experimentation, as follows:

A: a swab smear inoculum from an established culture, diluted in redistilled water in a 1:10 approx. wt. ratio (cell:water). Cell count was not done. A long, single smear was applied.

B: 0.5 ml of diluted inoculum just described above (SA) was spot-dropped in the center. Note: Dry spore inoculation was abandoned in the final trials because the resulting rapidity of germination would have required needlessly difficult measurement of very small time intervals.

6. Actual Experimental Results

The results in all trials proved positive, as expected in view of the preliminary trials. There were expected differences in vigor of the growths, in morphological patterns of micro-colonies, plus some aberrations from standard phenotype, but none pertinent to this invention.

The following findings have been clearly established:

A. the ionized zeolite inhibits Zn⁺⁺ activated bacterial proteases; and

B. the inhibition is substantially instantaneous.

7. Application of Zeolitic Enzyme Inhibitor

In some preliminary trials, the inhibitor was applied after a growth of micro-colonies was apparent under 10× magnification. This method was abandoned after it was well established that the inhibitor has an instant effect. Such instant effect is illustrated in FIG. 2. Also in some trials with fluid media, a similar delay in inhibitor application was adopted.

A. For the inoculation method 5A, pH 6.4 stabilized-deionized clinoptololite particles of mesh 200 (<74 μm) were applied onto one half of the plate (the other half serves as a control) immediately after inoculation: a. as a dust, and b. as a hydrosol.

B. For inoculation method 5A, pH 6.4 stabilized and deionized clinoptilolite particles of mesh 200 (<74 μm) were applied saturated in pieces of an inert porous absorptive material (filter paper) on the margin of the plate.

Note: In some preliminary runs, a bicomposite medium [FIG. 4] was also tried, by pouring one half of the plate in the original formulation, and the other half incorporating zeolitic inhibitor. Smearing inoculum over both halves of the plate, made the inhibitor effect immediately apparent.

The latter alternative also proved the tremendous adsorptive power of zeolites to transfer ions through gel substances, as for example mucus, covering GEC's (gas-exchanging cells) [FIG. 5] in alveoli and elsewhere, or even more importantly, the protective coats of microbes.

Preparation of this zeolite-containing alternative has some technical pitfalls. The zeolite must be incorporated cold into a warm agar to avoid an overly tight encapsulation of particles, and the generated vapor and condensed water conduce to inaccurate consistency of the medium.

8. Detailed Description of Drawing Figs.

FIG. 1: Enzyme coactors according to Holum

A complete enzyme (holoenzyme) consists of an apoenzyme (A) and one or more cofactors of the following three types: a, b, and c.

a. Coenzyme, a non-protein organic substance (e.g., a vitamin) which is dialyzable, thermostable, and loosely attached (single vertical connecting line) to the apoenzyme. It is a true substrate for enzyme-catalyzed reaction, and is recycled in a later step of a metabolic pathway by another enzyme.

b Prosthetic group, a dialyzable and thermostable organic substance. It is more firmly attached (multiple linking vertical connecting lines) to the protein of the apoenzyme portion.

c, Metal activator, a loosely attached metal cation, e.g., Zn⁺⁺, K⁺, Fe⁺⁺, Fe⁺⁺⁺, Ca⁺⁺, Mg⁺⁺, Co⁺⁺, Cu⁺⁺, or Mn⁺⁺. The metal cations are critical to the enzyme function, structure, and/or stability, and they ultimately are of great biological and medical importance.

FIG. 2: Adsorption of Enzyme Activator via Zeolite (H⁺ Concentration ˜10⁻⁷)

Hydrogen cations (H⁺) occupying some ion-exchangeable sites (S) on the exosurfaces and endosurfaces of zeolite Z are in equilibrium with H⁺ cations of the surrounding environment. When a bacterial digestive enzyme, a zinc-activated protease (DP), or a tripart toxin (comprising zinc-dependent lethal factor LF, plus oedema factor OF (an adenyl cyclase), plus four-domain protein PA, enters the adsorptive range of such a zeolite particle, zinc cations Zn⁺⁺ will be adsorbed, immediately inhibiting the bacterium and deactivating the toxin. Also deactivated will be toxins already excreted by the pathogens into the host macroenvironment (e.g., epithelium of alveoli, or bodily fluids.) Residual protein of the pathogens will be metabolized by the organism.

The drawing area designated Z represents only a minuscule part of the adsorptive surface of a zeolite particle, with interconnecting porous crystalline structure[18]. The small size of most zeolite pores (e.g., 4 Å, in the clinoptilolite example herein) precludes entry of pathogens or their proteins (e.g., bacterial digestive proteins, or toxins), whereupon rapid adsorption occurs on the outer surface, and (upon reaching equilibria there) thereafter proceeds within the zeolite particle.

FIG. 3: Petri dish with upper half untreated, lower half swab-smear Inoculated with B. mycoides. The upper half (A) of this plan view of a Petri dish of count agar exhibits (dark) thriving growth of micro-organisms, whereas its lower half (B), which was dusted with a zeolite provided according to this invention, remains essentially clear. The elapsed time was 34 hours after dusting of zeolitic inhibitor onto the lower half. The visible (patterned) antibiotic effect is attributable to the tendency of the resulting micro-colonies to grow “away from” the treated area.

FIG. 4: Petri dish with upper half untreated, lower half swab-smear Inoculated with B. cereus. A Petri dish, containing a modified nutrient agar (T-011)—resulting from assay investigation of alternative media—in both halves (A and B), plus zeolitic inhibitor in its lower half (B) only, was swab-smeared overall with B. cereus inoculum; shown as of 48 hours later. While, on the untreated half (A) bacteria (a) grew vigorously, only a smear residue (b) of the rapidly destroyed bacteria is apparent on the treated lower half (B). This shows the tremendous adsorptive capability of zeolites to transfer ions through gel-like substances, such as surfactants, in alveoli, and elsewhere, even more importantly the protective coats of microbes.

FIG. 5: Scanning Electron Micrograph (SEM) Image (5500×) Gas-Exchanging Surfaces

The SEM shows the Type II cells of the respiratory epithelium in a Pierophyllum sp. eleuterembryo, in principle identical with the Type II cells of alveolar epithelium in mammals. However, it is extremely difficult to prepare a specimum of alveolar epithelium surface without collapsing of the Type II cell surfaces. Accordingly, the undistorted surfaces of Type II cells in early embryonic stages of fish are useful for instant demonstration.

In humans, the germination of spores can take place anywhere in the respiratory system, as by inhalation, their final destination being on the squamous gas-exchanging cells of the alveolar epithelium. FIG. 5 “wrinkling” provides extensive gas-exchanging surface, which is covered with mucous surfactant as an effective protective membrane: Such a huge inner surface area in humans serves as a primary breeding ground for invading pathogens, where spores and early generations of vegetative cells establish themselves, until they massively reproduce and lyse through alveolar and capillary walls into the circulatory system. The alveolar surface is a critical site where intruding bacteria should be inhibited for protective treatment to be effective.

FIG. 6: Computer-Generated Image of Zeolite Open Structure of Crystals and Pores. [18]

Though computer-generated rather than “real” this image is impressively demonstrative or visually suggestive of the tremendous openwork surface area of solid zeolitic structures,

9. Mode of Inhibition

The negatively charged zeolite attracts and adsorbs the cation activators of enzymes [FIG. 2] which renders the enzymes (the bacterial digesting protease and toxin's LF protease) deactivated, The deactivated protein is then metabolized by the host organism.

10. Practical Applications of Zeolitic Enzyme Inhibitor

In all applications ion-adsorption is enhanced greatly in a wet environment. In a dry environment, e.g., on skin, inhibitor should be applied wet and be maintained wet. For therapy of cutaneous and intestinal infections by B. anthracis group bacteria, the use of clinoptilolite is entirely safe. For inhalational infection therapy, and in applications involving the circulatory system, a number of side effects of mechanical and biophysical nature should be considered.

B. Intestinal Infection may be Treated, as Follows:

a. By deionized zeolite administered orally, preferably before a meal, mixed in a drink (water, milk, tea) devoid of any salt preservative. USDA approved feed zeolite in feed is 2%. A substantially higher (more than 5 times the approved rate) may cause a temporary depletion of intestinal flora. Size of zeolite particles is not critical; 200 mesh (approx. 75 μm) being good. Of course, the smaller the particles are, the faster their adsorption will be. Zeolite passing through the digestive system deactivates bacteria and toxins; then it is excreted.

b. Following ingestion, deionized zeolite is at least partially H⁺ homo-ionized by the stomach acid, and then gradually stabilized to the equilibrium in the small intestine. Therefore, the zeolite may be administered encapsulated in any material soluble in a pH range 4.0-8.0.

C. Inhalational Infection

Practically, the germination of spores can take place anywhere in the respiratory system; however, the gas-exchanging cells of the simple squamous epithelium of alveoli constitute the final destination. Their wrinkled surface is covered with mucus, which serves as an effective filtering and protective membrane. This huge inner surface serves as the breeding ground of the invading bacteria, an epicentrum of the disease, where the spores and the early generations of vegetative cells gather, until they massively reproduce and lyse through the alveolar and capillary walls into the circulatory system. Therefore, the surface of alveoli is the optimal site for the bacteria to be inhibited, if a selected treatment is to have an optimal likelihood of success.

a. Preparation of dry zeolite in the very small particle size (<3 μm) necessary to reach the inner epithelium of alveoli is technically difficult. The most practical mode of administration is inhalation of a mist containing submicron particles, easily calibratable by sedimentation in a water column. Insofar as there is a concern about clogging of alveoli, as caused by an eventual extremely large overdose, even a complete coverage of the alveolar epithelium by zeolite particles delivered via aerosol (or hydrosol) would not inhibit in any appreciable extent the functioning of the epithelium. The zeolite's capacity for CO₂ adsorption is negligible, in view of the huge volume of CO₂ exchanged in the lungs. However, unlike a hydrosol, zeolitic dust in high concentrations, as in emergency use when water for appropriate preparation is not available, may temporarily desiccate the alveolar surface and hence may cause an extended need—expressed as coughing and temporary feeling of shortness of breath—for production of alveolar surfactant, as by type II cells. Even though only partial and temporary, such an administration process should be carefully monitored.

a. Preparation of dry zeolite in the very small particle size (<3 μm) necessary to reach the inner epithelium of alveoli is technically difficult. The most practical mode of administration is inhalation of a mist containing submicron particles, easily calibratable by sedimentation in a water column. Insofar as there is a concern about clogging of alveoli, as caused by an eventual extremely large overdose, even a complete coverage of the alveolar epithelium by zeolite particles delivered via aerosol (or hydrosol) of suitable composition (e.g., containing surfactant) would not inhibit in appreciable extent the functioning of the epithelium. The zeolite's capacity for CO₂ adsorption is negligible, in view of the huge volume of CO₂-exchanged in the lungs. However, unlike an a hydrosol, zeolitic dust in high concentrations, as in emergency use when water for appropriate preparation is not available, may temporarily desiccate the alveolar surface and hence may cause an extended need—expressed as coughing and temporary feeling of shortness of breath—for production of alveolar surfactant, as by type II cells. Even though only partial and temporary, such an administration process should be carefully monitored.

b. Except in utmost emergency, intravenous application should be avoided. Some specific concerns are obvious, such as deposition of zeolite particles in tissues. For an intravenous application, the particle size of the zeolite in the injected solution should be <1 μm, preferably close to the particle size in the colloidal suspension of the zeolite. In order to prevent an eventual calcium deficiency shock, or iron deficiency in hemoglobin, and similar cation-dependent problems, the deionized zeolites should be recharged with selected cations (e.g. calcium, iron, or magnesium cations).

c. In a hospital or similar setting, blood can be filtered (outside the body) through a bed of zeolitic inhibitor. This method is important in a situation when the toxins in blood reach an otherwise uncontrollable concentration. The particle size should be between 200-500 μm to allow free flow of blood and an instant effect, and be pretreated as described above in b. to prevent any eventual cation adsorption/exchange problems.

Also, zeolitic inhibitor may be selectively reionized by cation(s), which interfere with biochemical and biophysical processes in a pathogen (e.g., Ag⁺) or, more specifically, interfere with production or maintenance of protective coats of pathogens (e.g., Cu⁺⁺).

11. Prophylaxis

A. Dry and wet filters for gas masks, emergency homemade masks, mass transportation and building air filtration systems, etc.

B. Decontamination of skin and hair, clothing, homes and other enclosed or open areas.

C. Decontamination of drinking water and food, especially fruit and vegetables.

Key to Bracketed Numerical References:

[1] BOHINSKI (1973) Modern Concepts in Biochemistry, Allyn & Bacon, Boston.

[2] DRESSLER, David and Huntington POTTER (1991) Discovering Enzymes, Scientific American Library series, New York.

[3] WORTHINGTON, Von, Editor (1993) Worthington Enzyme Manual, Enzymes and Related Biochemicals, Worthington Biochemical Corporation, Lakewood, N.J.

[4] TABORSKY, Blanka E. (1986) Effects of Copper on Aquatic Organisms/Sublethal Toxicity of Cupric Cations on Early Developmental Stages of Some Fish. 37^{th} Intl Sci and Eng Fair (Fort Worth, Tex.}.

[5] TABORSKY, Blanka E. (1987) The toxicity of heavy metals and their removal from the aquatic environment. 38^{th} Intl Sci and Eng Fair (San Juan, Puerto Rico).

[6] CALLEGAN, Michelle (1998) Bacillus cereus endophthalmitis.

http://w3.ouhsc.edu/MPEIR/bacillus.html.

[7.] BEECHER, D J; Pulido, N P; Barney, N P; Wong, A C L. Extracellular virulence factors in Bacillus cereus endophthalmitis: Methods and implication of involvement of hemolysin BL. Infect Immun 63:632-639, 1995.

[8.] KLIMPEL, K R; Arora N; Leppia, S H. Anthrax toxin lethal factor contains a zinc metalloprotease consensus sequence which is required for lethal toxin activity, Molecular Microbiol 13:1093,1994.

[9] KETLER, J. M. et al. (1993) FEMS MICROBIOL. LETT., 111, 15-22.

[10] WREN, B. W.; Henderson, J. and L.; Ketley, J. M. (1994) Biotechniques, 16.7-8.

[11] TURNBULL, P C B (1981) Bacillus cereus toxins. Pharm. Ther. 13:453-505.

[12] PANNIFER, A. D. et al. (2001) Crystal structure—anthrax lethal factor, Nature 14.8.

[13] BRADLEY, Kenneth A. (2001) Identification of the cellular recipe for anthrax toxin. Nature 414:8.

[14] CHU, W P and T L Que, W K Lee, S N Wong. (2001) Minengoencephalitis caused by Bacillus cereus in a neonate. HKMJ 7(1):89-92.

[15] JAMA (1999) 281:1735-1745.

[16] DiPel®DF (2000) Material Safety Data Sheet

[17] TAYABAL, Azam F and Verner L Seligy (2000) Human Cell Exposure Assays of Bacillus thuringiensis Commercial Insecticides: Production of Bacillus Cereus-like Cytolytic Effects from Outgrowth of Spores, Enviro, Health Prospect 108:919-930.

[18] Zeolites 18/1, 1997, cover page.

[19] WORTHINGTON, Von, Editor (1993) Worthington Enzyme Manual, Enzymes and Related Biochemicals, 58, Worthington Biochemical Corporation, Lakewood, N.J.

Preferred embodiments and variants thereof have been suggested above for methods and procedures useful in practicing the present invention, as well as apparatus useful in methods so useful. Other modifications may be made, as by adding, combining, deleting, or subdividing any thereof, while retaining significant advantages and benefits of the invention, as defined in the following claims.

The claimed invention:

1. A zeolitic composition therapeutic against infection of a mammalian host by bacteria of the Bacillus anthracis Group: viz., B. anthracis, B. cereus, B. mycoides, B. thuringiensis.

2. Natural or synthetic zeolitic composition of claim 1, pretreated at least one of these ways:
a. at least partially deionized via equilibration by being washed with deionized or distilled water; b. H⁺ homo-ionized, by having cations at its ion-exchangeable sites replaced by hydrogen ions to an achievable maximum or other desired extent;
c. deionized by replacing its H⁺ cations at its ion-exchangeable sites replaced to desired extent by equilibration with hydroxyl group anions (OH⁻);
d. selectively (re)ionized by charging to desired extent with selected metallic cations.

3. Zeolitic composition of claim 2, comprising clinoptilolite in at least major part.

4. Zeolitic composition of claim 2, comprising colloidal particulate zeolite suitable for treating an infected host's exposed tissue so infected.

5. Method of therapeutically treating, by contacting, with zeolitic composition of claim 4 in liquid form on a cloth or similar applicator, such host's exposed tissue so infected.

6. Zeolitic composition of claim 2, of submicron size, in an aqueous or equivalent liquid mist, suitable for treating a host's infected lung(s) therewith, by the host's inhalation of such mist

7. Method of therapeutically treating, with aqueous mist containing such zeolitic composition of claim 6, such host's lung(s) so infected.

8. Zeolitic composition of claim 2, in a permeable bed of zeolitic particles about 200 to 500 microns in size, suitable for treatment of a host's infected blood by filtration via such bed.

9. Method of therapeutically treating, with such permeable filtration bed of such zeolitic particles of claim 8, such host's blood so infected.

10. Method of therapeutically treating a mammalian host for a tissue infection thereof caused by bacteria of the Bacillus anthracis Group (namely, B. anthracis, B. cereus, B. mycoides, or B. thuringiensis) by the step of applying to the infection site, in therapeutically appropriate manner, a zeolite theretofore H⁺ homo-ionized and remaining so until such application.

11. Method of preparing a zeolitic composition for therapeutic use against toxins produced by bacilli of the B. anthracis group, comprising the sequential steps of deionizing the zeolite, then reionizing the zeolite with selected cations, by washing it in one of the following ways, thus achieving (i) a preselected extent of removal thereof, or (ii) a preselected resulting pH:
a. with distilled or deionized water, and thereby removing by equilibration cations present initially upon exchangeable sites of the zeolite;
b. with dilute acid, and thereby replacing via ion-exchange cations initially present on ion-exchangeable sites of the zeolite;
c. with water having an electrolytically induced concentration of hydrogen ions;
d. with monovalent hydroxide or salt solution prior to such reionizing;
e. with water having a high concentration of hydroxyl group (OH⁻ anions) beforehand;
f. with water having a preselected concentration of metal cations Mⁿ⁺ (n=valence).

12. Method of applying a zeolitic composition of claim 11, comprising the step of bringing a zinc-activated bacterial protease into inhibiting intimate contact therewith, whereby Zn⁺⁺ cations are adsorbed by the zeolitic composition, deactivating the enzyme.

13. Method of applying a zeolitic composition of claim 11, comprising the step of inhibiting a tripartite toxin, having a zinc-dependent lethal factor, and a protective antigen (PA), whereby Zn⁺⁺ cations are adsorbed by the zeolitic composition, deactivating the toxin.

14. Method of applying a zeolitic composition of claim 11f, including also the step of inhibiting the bacteria by the step of adding Ag⁺ cations poisonous to said bacteria.

15. Method of applying a zeolitic composition of claim 11f, including also the step of inhibiting the bacterial production of their protective mucous coating, by the step of adding Cu⁺⁺ ions, thereby facilitating adsorption of Zn⁺⁺ cations from bacterial protease.

16 Method of applying a zeolitic composition of claim 11f, including the step of impregnating the zeolite with cobalt ions and thereby providing, in a situation of limited concentration of zinc cations and alternative metallic activator for carbon anhydrases, as enzymes essential to transport of CO₂, also accumulation and transfer of H⁺ and HCO₃.

17. Method of preventing, curing, or ameliorating indisposition of a mammalian host, said indisposition being attributable to exposure of the host to bacteria of a member of the Bacillus anthracis group, including the step of applying a zeolite to the member(s) of the host so affected or infected.

18. Apparatus for applying a suitably therapeutic zeolitic composition to preclude or to remedy a host's dysfunction attributable to bacteria of the Bacillus anthracis group, comprising
a. a porous cloth or paper applicator carrying suitably finely divided such composition;
b. an aerosol comprising gaseous or similarly suspending such composition;
c. a mist comprising water or other suitable suspending such composition; or
d. a filtrate comprising solution/suspension from a filtration bed of such composition.
